(19) United States
(12) Patent Application Publication
(10) Pub. No.: US 2020/0229918 A1
(43) Pub. Date: Jul. 23, 2020

(54) NOVEL TRANSCATHETER VALVE REPLACEMENT DEVICE

(71) Applicants: Thuy PHAM, East Windsor, CT (US); Caitlin MARTIN, East Windsor, CT (US); Qian WANG, East Winsor, CT (US)

(72) Inventors: Thuy PHAM, East Windsor, CT (US); Caitlin MARTIN, East Windsor, CT (US); Qian WANG, East Winsor, CT (US)

(21) Appl. No.: 16/486,753

(22) PCT Filed: Mar. 9, 2018
(86) PCT No.: PCT/US18/21873

§ 371 Date: Aug. 16, 2019

Related U.S. Application Data
(60) Provisional application No. 62/464,316, filed on Feb. 27, 2017.

(51) Int. Cl. A61F 2/24

(52) U.S. Cl. CPC A61F 2/2418; A61F 2002/828

(57) ABSTRACT

A heart valve leaflet replacement system for a diseased heart valve including a replacement valve that is configured to be selectively guided and implanted in a native annulus of the diseased heart valve. The replacement valve can include: a frame with a rigid portion to house and maintain the integrity of the replacement leaflets and a flexible portion enabling it to conform to the native vessel geometry, at least one prosthetic leaflet coupled to an inner surface of the stent, and a plurality of prong structures operatively coupled to and extending between portions of the at least one prosthetic leaflet and the inner surface of the bottom ventricular portion of the stent to selectively constrain the movement of the at least one prosthetic valve relative to the bottom ventricular portion of the stent.

### FIELD OF THE INVENTION

The application relates generally to replacement heart valves, preferably for replacing diseased pulmonary valve or aortic valve with valve insufficiency or stenosis. More particularly, embodiments of the subject matter related to tissue-based replacement heart valves and systems and methods to operatively deliver the replacement valve.

### BACKGROUND

Referring to FIG. 1, the pulmonary valve (PV) sits between the right ventricle and the pulmonary artery of a human heart and normally consists of three leaflets. Pathological alterations to the PV leaflets, PV annulus, or the right ventricular outflow tract (RVOT, such as, for example and without limitation, annulus or RVOT dilation, calcification, and leaflet thickening, can lead to altered PV function and cause PV insufficiency or stenosis. Similarly, the aortic valve (AV) sits in the aortic root between the left ventricle and the ascending aorta (AA) and normally consists of three leaflets. Pathological alterations to the AV leaflets, AV annulus, sinotubular junction (STJ), ascending aorta or the LVOT, such as, for example and without limitation, dilation of annulus, root, STJ or AA, calcification, and leaflet thickening, can lead to altered AV function and cause AV insufficiency or stenosis.

PV insufficiency is dysfunction of the PV related to improper coaptation of the leaflets during diastole that causes an abnormal leakage of blood from the pulmonary artery back into the right ventricle. PV stenosis is dysfunction of the PV related to improper opening of the leaflets during systole that obstructs normal blood flow from the right ventricle to the pulmonary artery. PV dysfunction commonly presents in patients with congenital heart defects (CHD). AV insufficiency is dysfunction of the AV related to improper coaptation of the leaflets during diastole that causes an abnormal leakage of blood from the aortic artery back into the left ventricle. AV stenosis is dysfunction of the AV related to improper opening of the leaflets during systole that obstructs normal blood flow from the left ventricle to the aorta. AV dysfunction commonly presents in patients with congenital heart defects (CHD) such as bicuspid aortic valve, and/or often associated with calcification and advanced age.

Children with complex CHDs involving the RVOT, including defects such as tetralogy of Fallot, truncus arteriosus, valvular pulmonic stenosis, and transposition of the great arteries, typically undergo surgical repair in the first days or months of life. The RVOT of these patients is usually reconstructed surgically by the use of a transannular patch or a valved right ventricle-to-pulmonary artery (RV-PA) conduit.

Due to the nonliving nature of such conduits (composed of either synthetic material or nonviable homograft or xenograft tissue), RVOT dysfunction, such as stenosis and insufficiency (FIG. 2), occur over time due to the development of calcification, intimal proliferation, and graft degeneration. Subsequently, most cases require surgical conduit revisions within 10 years, and multiple open heart operations over the patient's lifetime.

Transcatheter pulmonary valve (TPV) replacement (TPVR) was first reported in an RVOT conduit in 2000 as a means of delaying eventual surgical conduit replacement. Today, it has become an accepted and practiced treatment method for dysfunctional RVOT. The less invasive TPVR holds significant advantages over the surgical approach due to fewer hospital days and less traumatic injury to patients.

Currently, the Medtronic Melody valve is an example of TPVs approved by the US Food and Drug Administration (FDA) for the treatment of adult and pediatric patients who suffer from either a narrowed pulmonary valve or moderate or greater pulmonary regurgitation caused by CHD. TPVR with the Melody valve has shown good hemodynamic and clinical outcomes up to 7 years after implantation; and there have been 10,000 Melody valve implants worldwide.

The current Melody valve (which is available in 2 sizes, Melody TPV 20 and TPV 22) was designed to treat patients with dysfunctional RVOT conduits ≤24.5 mm in diameter, which only account for approximately 15-30% of patients with CHD in whom pulmonary valve replacement is indicated. There is a large number of patients with a dysfunctional native non-circular or transannular-patched RVOT who are not suitable for the current Melody valve. It is anticipated that a transcatheter valve designed for larger RVOTs will serve approximately three to four times as many patients as the current Melody TPV. Secondly, the Melody valve's 22 Fr delivery system remains rather large and the long stent frame can be difficult to implant in smaller pediatric patients.

The pulmonary trunk anatomical geometry and potential lack of calcification pose challenges for TPV delivery and anchoring. The surgically implanted transvalvular patches often become significantly dilated over time, and a TPV device deployed within this region may cause further dilation. Furthermore, the RVOT and pulmonary trunk geometry can vary significantly among patients.

Accordingly, it would be beneficial to have a heart valve leaflet replacement system that does not suffer from the shortcomings and deficiencies of conventional valve prosthetics. It is desirable to secure the prosthetic pulmonary valve replacement system to the native pulmonary annulus and/or transvalvular graft. It is also desirable to improve positioning of a TPV and prevent leaking of blood between the TPV and the native pulmonary root and/or graft. Similarly, it is desirable to retain the TPV device during the cardiac cycle and prevent further dilation of the surrounding tissue, i.e. pulmonary artery, annulus, RVOT, graft, etc. Furthermore, other desirable features and characteristics will become apparent from the subsequent detailed description and the appended claims, taken in conjunction with the accompanying drawings and the foregoing technical field and background.

### BRIEF SUMMARY OF THE INVENTION

Described herein is a heart valve replacement device that can be implanted to restore normal function of a diseased heart valve. In one aspect, the heart valve replacement system can be configured to secure the replacement heart valve to the native pulmonary or aortic root. For clarity, it will be appreciated that this disclosure will focus on the treatment of PV or RVOT dysfunction, however it is contemplated that the heart valve replacement system and the associated methods can be used or otherwise configured to be used to treat other valve disease conditions and replace other valves of the human heart, or could be used or otherwise configured to be used in other mammals suffering from valve deficiencies as well.

In one aspect, the heart valve replacement system can comprise a prosthetic PV that is configurable or otherwise sizeable to be radially crimped down to fit within a delivery sheath and to subsequently be selectively expanded to an operative size once removed from the delivery sheath within the heart. In a further aspect, at least a portion of the replacement prosthetic PV can have a stent shape, which can comprise an upper flared portion, a rigid middle portion, at least one bendable portion, one adjustable length portion, and a lower flared portion.

In one aspect, the upper flared portion can be configured to facilitate anchoring of the stent in the pulmonary artery or pulmonary bifurcation, and the lower flared portion can be configured to facilitate anchoring of the stent in the RVOT, which can help prevent paravalvular leakage and dislodgement of the stent, the rigid middle portion can house at least one prosthetic leaflet, and the bendable portions can conform the native pulmonary geometry.

In one aspect, the heart valve replacement device can be configured to have a similar compliance as the native pulmonary artery in order to impose optimal radial expansion force on the native vessel for anchoring the device without causing further dilation of the surrounding tissue.

In one aspect, the heart valve replacement device can be configured to have an adjustable length to facilitate device anchoring in the RVOT and pulmonary bifurcation in a wide range of patients which have different pulmonary artery anatomies. In one exemplary aspect, the stent could have at least two segments which are not rigidly connected to allow for length adjustment.

It is further contemplated that at least some portion of the stent could be configured to be flexible or bendable in order to better conform to the native vessel geometry when implanted to aid in preventing leakage of blood between the operatively positioned PV prosthesis and the native PV. In one exemplary aspect, the middle portion of the stent housing the replacement prosthetic valve could be configured to be rigid (i.e., non-bendable), to ensure that the valve configuration is unpertubed and circular in profile, which is important for proper valve function and durability.

In one aspect, the replacement prosthetic PV can comprise a skirt that can be coupled to at least a portion of the inner lumen of the stent and/or to at least a portion of the outer side of the stent.

In one aspect, at least one prosthetic leaflet can be mounted on the inner lumen of the stent. In this aspect, each leaflet of the prosthetic replacement valve can have a pronged shape with at least one prong which reduces the peak stress acting on the leaflet, and maintains proper coaptation. By reducing the peak stress on the leaflet, the leaflet durability can be enhanced. In a further aspect, with the reduction in peak stress, the leaflet thickness can be reduced to facilitate a smaller device crimped profile important for patient safety particularly for small and pediatric patients, without compromising leaflet durability.

In one aspect, it is contemplated that the at least one prosthetic leaflet can have an extended free edge and/or prongs to facilitate leaflet coaptation when the stent is over expanded. In a further aspect, the extended free edge and/or prongs can be designed to maintain leaflet coaptation and replacement PV competency even if the stent is further dilated, for example from size 23 mm to size 25 mm, after the initial implantation. In this aspect, the replacement PV device can accommodate for dilation of the main pulmonary artery seen in patients several years after the initial implantation. It is further contemplated that after several years of implantation of the PV, the pediatric or young patient's pulmonary root and pulmonary artery can grow in size (i.e, a larger diameter). The replacement PV device can be expanded by, for example, self expansion or a transcatheter balloon, from size 23 mm to size 25 mm, to accommodate the patient growth. Due to the reserved free edge length and/or prongs at the initial configuration during the initial implantation, the replacement PV device can remain competent post device dilation.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 is a schematic view of the normal pulmonary valve anatomy and its location within the human heart.

FIG. 2A is an illustrated perspective of a pulmonary conduit which has narrowed and become stenosed.

FIG. 2B is an illustrated perspective of a pulmonary conduit in which the leaflets can no longer coapt resulting in regurgitation of blood back into the right ventricle.

FIG. 3 shows one embodiment of the heart valve replacement system.

FIG. 4 shows one embodiment of the stent design of the heart valve replacement system.

FIG. 5 shows one embodiment of the stent design, demonstrating length adjustment of the stent along the axial direction of the stent by adjusting length-adjustable portions.

FIG. 6A shows the stent design with one embodiment of a bridge connection between the stent segments with the bridge connection is a straight section connecting from one joint to another joint of two separate stent segments.

FIG. 6B shows the stent design with one embodiment of the bridge connection where it is a straight section connecting one strut to another, such that the location of bridge connections can vary along the stent circumference at different stent segments.

FIG. 6C shows the stent design with one embodiment of the bridge connection where it is a curved section connecting one strut to another, such that the location of bridge connections can vary along the stent circumference at different stent segments.

FIG. 7 shows one embodiment of the leaflet and prong design of the heart valve replacement system.

FIG. 8 shows one embodiment of the 2D stent-leaflet attachment curves and stent-prong attachment points for a size 23 mm heart valve replacement system.

FIG. 9 shows one embodiment of the 3D stent-leaflet attachment curves and stent-prong attachment points for a size 23 mm heart valve replacement system.

FIG. 10A illustrates an attachment of a prosthetic leaflet and prongs to the stent.

FIG. 10B illustrates that stent struts can be configured with holes to facilitate attachment of the prongs.

FIG. 11 shows a physical prototype of one embodiment of the heart valve replacement system.

FIG. 12A shows an exemplary pulmonary root anatomical structure and its curvature.

FIG. 12B shows a physical pulmonary root anatomical structure and its curvature.

FIG. 12C shows a physical prototype of one embodiment of the heart valve replacement system implanted within a 3D printed replica of the native pulmonary artery geometry demonstrating that the heart valve replacement system can bend to conform to the native geometry of the pulmonary artery.

FIG. 13A shows the valve closed geometries for the physical prototype in FIG. 11 in hydrodynamic tests specified in ISO 5840-3:2013 Cardiovascular implants—Cardiac valve prostheses—Part 3: Heart valve substitutes implanted by transcatheter techniques.

FIG. 13B shows the valve open geometries for the physical prototype in FIG. 11 in hydrodynamic tests specified in ISO 5840-3:2013 Cardiovascular implants—Cardiac valve prostheses—Part 3: Heart valve substitutes implanted by transcatheter techniques.

FIG. 14 shows at least one prosthetic leaflet with an extended free edge that can be designed to span the width of the leaflet free edge to ensure sufficient coaptation between the leaflets.

FIG. 15 shows one two dimensional depiction of a section of a leaflet and prong that can have the stent-leaflet attachment line 24/25, and stent-prong attachment points 23.

FIG. 16 shows a three dimensional depiction of a section of a leaflet and prong that can have the stent-leaflet attachment line 24/25, and stent-prong attachment points 23.

FIG. 17A shows a first embodiment of the attachment of a leaflet and prongs to the stent at a 3D stent-leaflet attachment point as disclosed herein.

FIG. 17B shows a second embodiment of the attachment of a leaflet and prongs to the stent at a 3D stent-leaflet attachment point as disclosed herein.

FIG. 18A illustrates a top cross section view of an attachment of a prosthetic leaflet to a stent via attachment tabs as described herein.

FIG. 18B illustrates a side view of an attachment of a prosthetic leaflet to a stent via attachment tabs as described herein.

### DETAILED DESCRIPTION OF THE INVENTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, and, as such, can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

For clarity, it will be appreciated that this disclosure will focus on the treatment of PV and/or RVOT dysfunction, however it is contemplated that the heart valve leaflet replacement system and the associated methods can be used or otherwise configured to be used to replace other valves of the human heart, or could be used or otherwise configured to be used in other mammals suffering from valve deficiencies as well.

As used throughout, the singular forms “a,” “an” and “the” include plural referents unless the context clearly dictates otherwise. Thus, for example, reference to “a leaflet” can include two or more such leaflets unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and also includes any combination of members of that list. Further, one should note that conditional language, such as, among others, “can,” “could,” “might,” or “can,” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular embodiment.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these cannot be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems can be understood more readily by reference to the following detailed description of preferred embodiments and the examples included therein and to the Figures and their previous and following description.

Described herein is a heart valve replacement system 7 that can be implanted in one of the native annuli. In one aspect, it is contemplated that the heart valve replacement system 7 and the associated methods can be configured to secure the replacement heart valve to the pulmonary annulus and/or pulmonary graft. In a further aspect, the heart valve replacement system 7 and the associated methods can be configured to secure the implanted pulmonary prosthesis during a cardiac cycle and help restore normal function of the pulmonary valve (PV) 1. It should be noted that it is contemplated that the heart valve replacement system 7 described herein can be used to replace any diseased valve within the heart. For illustrational purposes, the description for this invention is focused on the pulmonary valve, and naming is done according to the pulmonary anatomy. However, all other heart valves will have similar structures so that the designs described herein can be used accordingly.

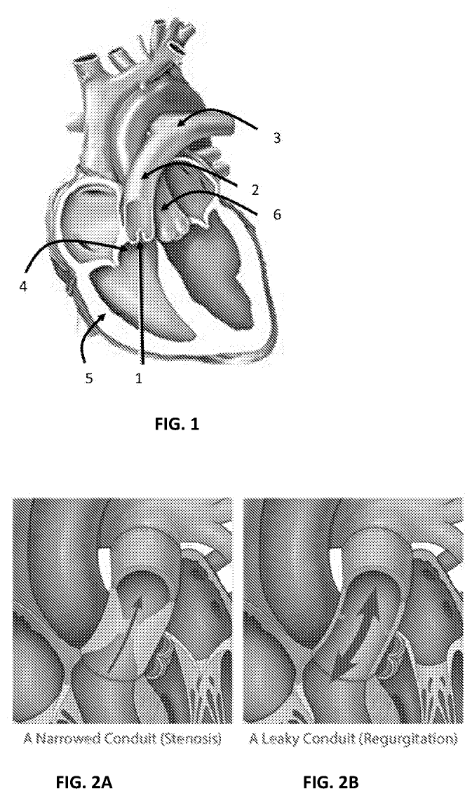

Referring to FIG. 1, the PV 1 is located on the right side of the heart between the pulmonary artery 2 and the right ventricle (RV) 5 and has three leaflets. The portion of the RV 5 proximal to the PV 1 is referred to as the RVOT 4. The main pulmonary artery 2 extends from the pulmonary valve 1 to the pulmonary bifurcation 3. The aortic root 6 sits between the left ventricle and ascending aorta.

Referring to FIG. 2, right ventricle-pulmonary artery conduits to treat RVOT dysfunction often fail several years after implantation. In one aspect, exemplified in FIG. 2A, the conduit can become stenosed, and in another aspect, exemplified in FIG. 2B, the conduit can become regurgitant. Patients treated with transannular patches or grafts to treat RVOT dysfunction also often redevelop RVOT dysfunction after several years.

In one aspect, referring to FIG. 3, disclosed herein is an exemplary aspect of the heart valve replacement system 7, which can comprise at least one prosthetic leaflet 8, at least one prong structure 9, and a rigid stent 10, where the prong structure 9 operatively couples a portion of the prosthetic leaflet 8 to a portion of the stent 10. Further, in this aspect, the heart valve replacement system 7 can comprise at least one bendable portion 11 which can aid the heart valve replacement system 7 conform to the native pulmonary artery 2 when operatively positioned, and at least one adjustable length portion 15 which can be adjusted to permit optimal sizing in variable native PA geometries.

In one aspect, the replacement prosthetic valve 7 can be configured to be selectively compressed or otherwise constrained to a compressed position, in which replacement prosthetic valve 7 has a reduced diameter that is suitably sized to allow for operative positioning of the replacement prosthetic valve 7 within a delivery catheter. The replacement prosthetic valve 7 is also configured to allow for selective expansion of the replacement prosthetic valve 7 to an expanded operative position once the replacement prosthetic valve 7 is selectively positioned in the desired location within the heart.

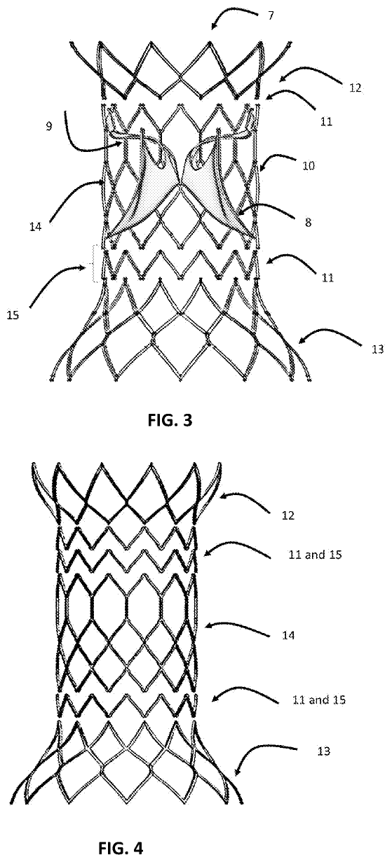

In a further aspect, as exemplified in FIG. 4, the heart valve replacement system stent can comprise an upper flared portion 12, a lower flared portion 13, a cylindrical middle portion 14, a bendable portion 11, and a length adjusting portion 15. In this aspect, it is contemplated that the upper flared portion 12 be configured to aid in anchoring the heart valve replacement system 7 in the pulmonary artery 2 and/or the pulmonary bifurcation 3, and the lower flared portion 13 to aid in anchoring the heart valve replacement system 7 in the RVOT 4, to prevent dislodgement of the device and prevent paravalvular leakage. In one aspect, the upper flared portion 12 and lower flared portion 13 can have a partial toroid shape to mimic the complementary surface of the pulmonary bifurcation 3 and RVOT 4 respectively. Optionally, it is contemplated that the upper flared portion 12 and lower flared portion 13 can have a conical or cylindrical shape.

In exemplary aspects, the upper flared portion 12 can be configured to adapt or otherwise conform to the native pulmonary artery 2 or bifurcation 3, the lower flared portion 13 can be configured to adapt or otherwise conform to the native RVOT 4, the rigid middle portion 10 can be configured to adapt or otherwise conform to the native valve 1 and/or pulmonary artery 2, and the at least one bendable portion 11 and length adjusting portion 11 can be configured to adapt or otherwise confirm to the native pulmonary artery 2, such that, for example, the heart valve replacement system 7 can be shaped, bended, extended, and positioned as desired to facilitate anchoring, fixating, and sealing.

In one aspect, the bendable portion 11 of the heart valve replacement system 7 can be configured to attach to the other portions of the stent with fewer or no rigid connections, such that, the heart valve replacement system 7 can bend at this region. In this aspect the heart valve replacement system 7 can be configured to conform with a wide range of patient native pulmonary artery geometries, which can aid in device anchoring and can prevent leakage between the heart valve replacement system 7 and the surrounding tissue when operatively positioned. In a further aspect, the bendable portion 11 and length adjusting portion 15 can be configured to have a cylindrical, conic, and/or partial toroid shape.

Referring to FIGS. 4 and 5, in one exemplary aspect, the bendable portion 11 can be configured as a length adjusting portion 15 by utilizing an open mesh structure between adjacent stent portions. In this aspect, disconnected stent portions can be connected by, for example, the sealing component such as fabric, PET, PTFE, polyester cloth and/or pericardial tissues, which allows the heart valve replacement system 7 to bend. Further in this aspect, the disconnected nature of the stent can be configured to allow for length adjustment of the heart valve replacement system 7, such that the heart valve replacement system 7 can be adjusted to better fit a particular patient's pulmonary artery anatomy which can vary in length. In one exemplary aspect, the bendable portion 11 and length adjusting portion 15 can be configured as a gap in the stent 16 about 1-10 mm in length to allow for 1-10 mm length adjustment of the heart valve replacement system 7. For example, the heart valve replacement system 7 can be shortened by adjusting the gap in the stent 16 following the arrows in FIG. 5.

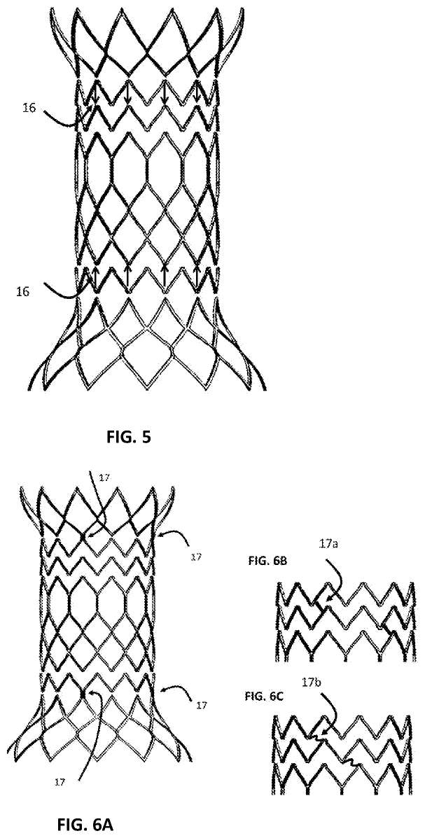

Referring to FIG. 6, in one aspect, it is contemplated that the bendable and length adjusting portions 11/15 of the heart valve replacement system 7 can have a series of sparse vertical links between struts of stent cells at different heights/rows. In one exemplary aspect, 3-4 links 17 of various shapes can be formed, symmetrically or asymmetrically around the circumference of the stent, between two rows of the stent cells, which allows for easy bending of the heart valve replacement system 7 to conform to the native vessel curvature. The shape of the links can be a straight line 17a as shown in FIGS. 6A and 6B or a curved line 17b as shown in FIG. 6C that permits easy bending and length adjustment of the heart valve replacement system 7 through either plastic or elastic deformation of the links 17.

In one aspect, it is contemplated that the bendable and length adjusting portions 11/15 of the prosthetic valve 7 can be configured from different materials than the stent material, such as as fabric, PET, PTFE, polyester cloth and/or pericardial tissues, which can be folded or compressed to reduce the total length/height of the heart valve replacement system 7, or unfolded or elongated to increase the total length/height of the heart valve replacement system 7. It is further contemplated that the adjustment of the length adjusting portion 15 can be done pre-operatively based on the known patient anatomy or intra-operatively based on the anchoring and deployment of the heart valve replacement system 7.

Referring to FIG. 3, in one aspect, the middle portion can have a cylindrical or conical shape. It is further contemplated that the at least one prosthetic leaflet 8 be coupled to the middle portion 14. In one exemplary aspect, the middle portion 14 can have a height range of between about 0.5 to about 1.5 times the radial length of the displaced diseased leaflets. In this aspect, the middle portion 14 can displace diseased native leaflets and/or pulmonary graft or conduits out of the blood flow tract upon expansion to an operative position. Furthermore, in this aspect, the middle portion 14 can be configured to be rigid (i.e. non-bendable) to prevent distortion of the at least one prosthetic leaflet 8, which is important for the heart valve replacement system 7 function and durability.

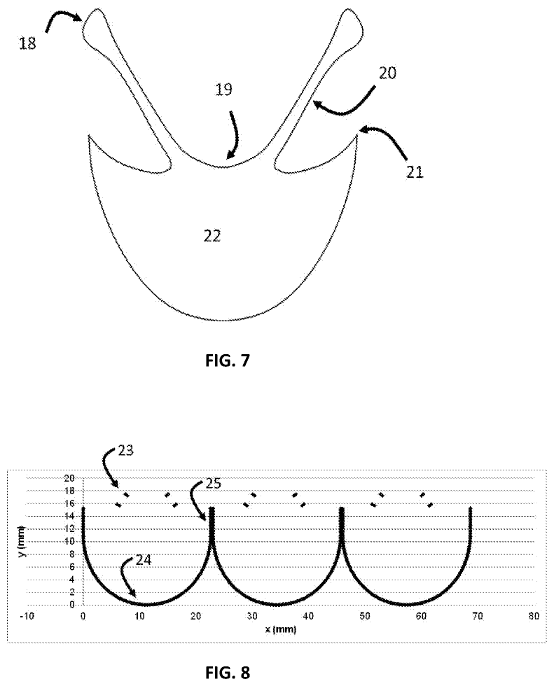

In one aspect, it is contemplated that the middle portion 14 of the heart valve replacement system 7 can act as a replacement prosthetic valve by itself. It is contemplated that given suitable patient anatomies (e.g. short and non-cylindrical shaped pulmonary trunk), the middle portion 14 can be deployed and operatively positioned in patients on its own to treat diseased valves, without the upper 12 and lower 13 flare stent portions for anchoring. It is further contemplated that the middle portion 14 can be selectively designed to couple with the specific leaflet shape illustrated in FIG. 7 that has an extended free edge 19, prongs 20 with stent attachment tabs 18, high commissures 21, and a belly 22. In its operative deployed position, functioning as an individual valve, the middle portion 14 can be oversized by 10% to 20% with respect to the size (i.e., diameter) of native vessel, root, or valve annulus to prevent dislodgement of the device and leakage between the device and vessel.

Referring to FIG. 7, in one aspect, it is contemplated that the at least one prosthetic leaflet 8 can have an extended free edge and/or prongs 9 to facilitate leaflet coaptation when the stent is over expanded. In a further aspect, the extended free edge and/or prongs 9 can be designed to maintain leaflet coaptation and replacement valve competency even if the stent is further dilated, for example from size 23 mm to size 25 mm, after the initial implantation. In this aspect, the heart valve replacement system 7 can accommodate for dilation of the main pulmonary artery 2 seen in patients several years after the initial implantation. It is further contemplated that after several years of implantation of the PV, the pediatric or young patient's pulmonary root and pulmonary artery can grow in size (i.e, a larger diameter). The heart valve replacement system 7 can be expanded by, for example, a transcatheter balloon, from size 23 mm to size 25 mm, to accommodate the patient growth. Due to the reserved free edge length and/or prongs 9 at the initial configuration during the initial implantation, the replacement PV device can remain competency post device dilation.

In one embodiment of the valve as depicted in FIG. 14, the at least one prosthetic leaflet 8, the extended free edge 19 can be designed to span the width of the leaflet free edge to ensure sufficient coaptation between the leaflets.

In one embodiment of the middle portion 14, the stent can have a height ranging from 18 to 20 mm, and is comprised of cobalt chromium material with a stent strut width and thickness of 0.40 mm and 0.35 mm, respectively. In one embodiment of the middle portion 14, shown in FIG. 10, the stent strut is designed with one or two prong attachment holes 26 to facilitate the attachment of the prongs 9 to the stent. It is contemplated that the attachment of the prongs can be realized using a variety of means, including, but not limited to, heat, chemical (such as adhesives), or mechanical (such as suture) bonding, at various parts of the heart valve replacement system 7, including but not limited to, the upper flare portion 12.

In one embodiment of the middle portion 14 illustrated in FIG. 17, the stent can have an outer diameter of about 25 mm and a height ranging from 25 to 30 mm, and is comprised of self-expandable Nitinol material with a stent strut width and thickness of about 0.30 mm and 0.35 mm, respectively. The stent parameters were scientifically optimized such that the stent can be crimped into a 12-Fr delivery catheter without damaging the stent, i.e. the stent strains determined through finite element analysis were within 12% which is within the elastic regime of Nitinol. The stent parameters were also scientifically optimized for implantation into the main pulmonary artery. Stent deployment into a porcine main pulmonary artery with an inner diameter of 19 mm was simulated using finite element analysis. The stent expanded the vessel from 19 mm to 24 mm and imposed a radial contact force of approximately 13 N on the vessel wall, which is sufficient to anchor the stent in place, while minimizing the likelihood of damage to the vessel wall, further vessel dilation, and obstruction of the surrounding heart structures.

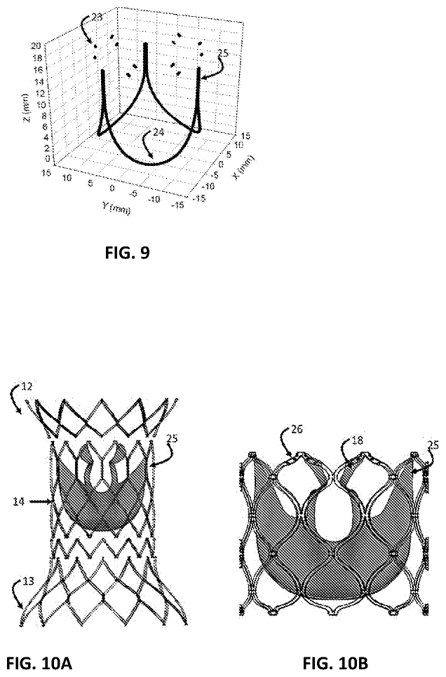

In one aspect, it is contemplated that the leaflet 8 and prong 9 can be designed to reduce leaflet stress during valve closure and prevent the leaflet hitting any portion of the heart valve replacement system 7 during valve opening. In one embodiment, for a middle portion stent with an outer diameter of 23 mm and height of 18 mm, the leaflet 8 as illustrated in FIG. 7 can have a maximum width of 25 mm and height of 14 mm, where the extended free edge portion is 0.5 to 1.0 mm higher than the lower regions of the free edge, and the prong structures 9/20 can have an angle of between 45 to 55° and a length of between 12 to 17 mm extending from the free edge of the leaflet. Further in this embodiment, the leaflet 8 and prong 9/20 can have the stent-leaflet attachment line 24/25, and stent-prong attachment points 23 specified in 2D in FIG. 8 and in 3D in FIG. 9. Illustrated in FIG. 10 is the attachment of the leaflet 8 and prongs 9/20 to the stent at the 3D stent-leaflet attachment points 24/25 and stent-prong attachment points 23 respectively. In one embodiment shown in FIG. 10, the stent-prong attachment points 23 can align with prong attachment holes 26 in the stent to facilitate attachment. This specific leaflet and prong design and its variations (+/−25% derivation from the illustrated design curves) were scientifically optimized for optimal leaflet stress reduction during the cardiac cycle while having a large effective orifice area without hitting the stent during valve opening (FIG. 8). This design can be scaled, or proportionally adjusted, or un-proportionally adjusted for different sizes of the valve, provided these structures of the leaflet 8 and prongs 9 are used for the purpose of reducing leaflet stress and increasing valve durability.

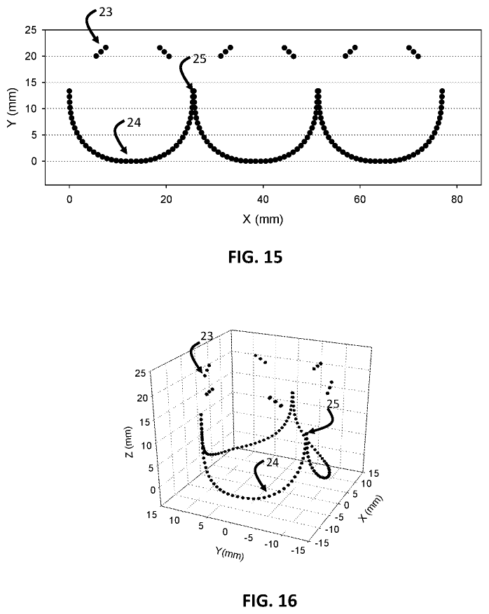

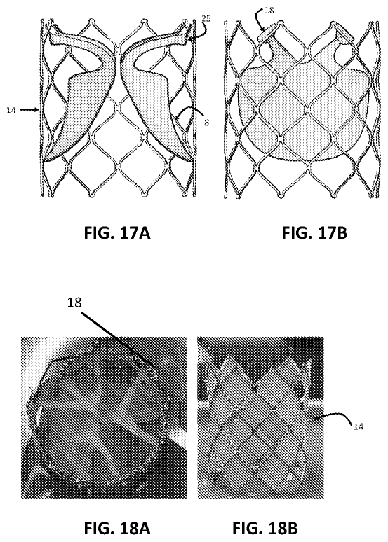

In one aspect, it is contemplated that the leaflet 8 and prong 9 can be designed to reduce leaflet stress during valve closure and prevent the leaflet hitting any portion of the heart valve replacement system 7 during valve opening. In one embodiment, for a middle portion stent with an outer diameter of 25 mm and a height of approximately 30 mm, the leaflet 8 as illustrated in FIG. 14 can have a maximum width of 24 mm and height of 22 mm, where the extended free edge portion is 4.0 to 8.0 mm higher than the commissures, and the prong structures 9/20 can have an angle of between 45 to 55° and a length of between 12 to 17 mm extending from the free edge of the leaflet. Further in this embodiment, the leaflet 8 and prong 9/20 can have the stent-leaflet attachment line 24/25, and stent-prong attachment points 23 specified in 2D in FIG. 15 and in 3D in FIG. 16. Illustrated in FIG. 17 is the attachment of the leaflet 8 and prongs 9/20 to the stent at the 3D stent-leaflet attachment points 24/25 and stent-prong attachment points 23 respectively. In one embodiment shown in FIG. 17, the stent-prong attachment points 23 can align with the stent struts to facilitate attachment. This specific leaflet and prong design and its variations (+/−25% derivation from the illustrated design curves) were scientifically optimized for optimal leaflet coaptation under physiological pulmonary blood pressure, while maintaining low leaflet stress, as well as a large effective orifice area without hitting the stent during valve opening. This design can be scaled, or proportionally adjusted, or un-proportionally adjusted for different sizes of the valve, provided these structures of the leaflet 8 and prongs 9 are used for the purpose of reducing leaflet stress and increasing valve durability.

In one aspect, at least a portion of the heart valve replacement system 7 can be covered with a sealing component to help to prevent paravalvular leakage after implantation, which can be attached via conventional means, such as, for example and without limitation, sewing, medical grade adhesives, and the like. It is further contemplated that the upper and lower flared portions 13, middle portion 14, bendable 11 and length adjusting 15 portions of the stent can be formed from the same or different materials.

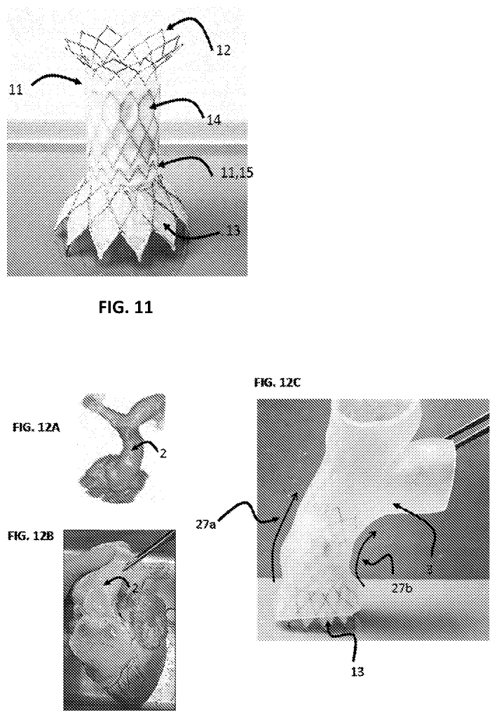

Referring to FIG. 11, in one exemplary aspect, the heart valve replacement system 7 can be comprised of the middle section of the frame only. In this aspect, the stent can be comprised of a Nitinol stent covered with a porcine pericardial skirt to prevent the leakage of blood between the device and surrounding tissue when implanted in the operative position.

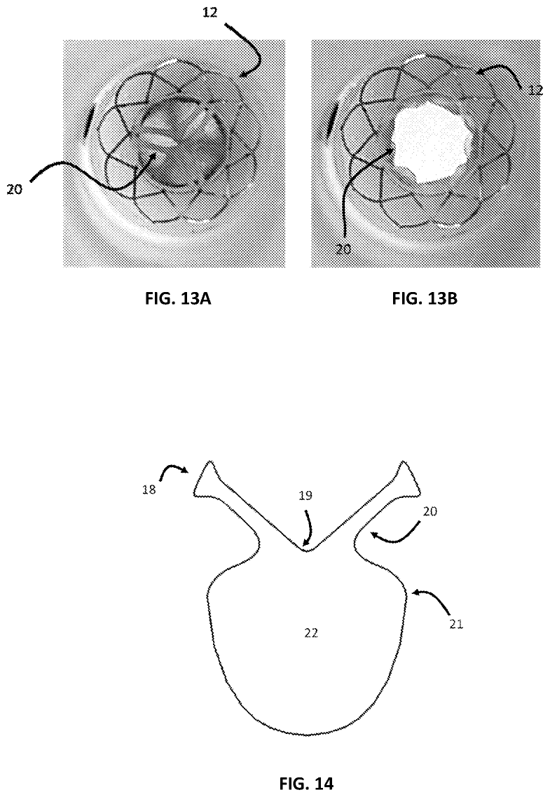

Referring to FIG. 11, in one exemplary aspect, the heart valve replacement system 7 can be comprised of a Nitinol stent covered with a skirt at the lower flare 13, middle section 14, and bendable/length adjusting section 11/15 to prevent the leakage of blood between the device and the surrounding tissue when implanted in the operative position. Further in this aspect, the heart valve replacement system 7 can be left uncovered at the upper flare 12 so as to not perturb blood flow from the valve to the pulmonary artery 2. Further in this aspect, referring to FIG. 12, the bendable/length adjusting section 11/15 permits the heart valve replacement system 7 to conform with the native RVOT/pulmonary artery anatomy. Shown in FIG. 12A is a representative pulmonary artery 2 geometry, demonstrating the native pulmonary artery 2 is curved. FIG. 12B is the physical pulmonary artery 2 of an animal where the pulmonary vessel 2 is also curved. FIG. 12C demonstrates that the fabricated prototype heart valve replacement system 7 can conform to a 3D printed pulmonary artery replica model at both the inner 27b and outer 27a vessel curvatures. The heart valve replacement system 7 was also subjected to in vitro hydrodynamic tests specified in ISO 5840-3:2013 Cardiovascular implants—Cardiac valve prostheses—Part 3: Heart valve substitutes implanted by transcatheter techniques. Referring to FIG. 13, the exemplary heart valve replacement system 7 design can close and open properly under pulmonary diastolic and systolic pressures respectively.

In one aspect, it is contemplated that the stent of the heart valve replacement system 7 can be formed using conventional stent forming and fabrication methodologies and stent configurations. In this aspect, at least a portion of the upper 12 and lower 13 flared portions and/or a portion of the middle portion 14 can be formed to be self-expandable or balloon-expandable to the desired operative positon. In this aspect, it is contemplated that the stent can be laser cut or woven into a desired conventional stent design that can be radially collapsible and expandable. Thus, it is contemplated that the stent can comprise a plurality of operatively linked components that form an expandable meshed body that can be formed from a metal, such as, for example and without limitation, cobalt chromium, stainless steel and the like; or a metal having inherent shape memory properties, such as, for example and without limitation, Nitinol and the like.

What is claimed is:

1. A transcatheter valve replacement device comprising:
a valve frame comprising adjustable portions to accommodate a native curvature of an implantation site,
wherein the adjustable portions support a plurality of prosthetic leaflets within the valve frame such that the valve frame corrects the implantation site to a proper curvature; and
wherein at least one of the prosthetic leaflets comprises an extended leaflet free edge and at least one prong structure that couples the at least one prosthetic leaflet to a respective structural component of the valve frame.

2. A transcatheter valve replacement device according to claim 1, wherein the adjustable portions of the valve frame in conjunction with the at least one prosthetic leaflet are positioned within the implantation site such that the transcatheter valve replacement device exhibits a minimized leaflet stress during a cardiac cycle.

3. A transcatheter valve replacement device according to claim 2, wherein the valve frame supporting the plurality of prosthetic leaflets maintains the proper curvature of the implantation site over a range of diameters across which the implantation site expands during the cardiac cycle.

4. A transcatheter valve replacement device according to claim 1, wherein the valve frame defines an upper flared portion, a lower flared portion, a middle portion, a bendable portion, and a length adjusting portion, or combinations thereof.

5. A transcatheter valve replacement device according to claim 4, wherein the upper flared portion is configured to anchor the valve frame in a pulmonary artery and/or a pulmonary bifurcation, and wherein the lower flared portion is configured to anchor valve frame in the right ventricular outflow tract (RVOT).

6. A transcatheter valve replacement device according to claim 4, wherein the upper flared portion, lower flared portion, the bendable portion, and length adjusting portion each have a respective partial toroid shape or a conical shape or a cylindrical shape.

7. A transcatheter valve replacement device according to claim 4, wherein the bendable portion is configured to attach to at least one of the upper flared portion, the lower flared portion, the middle portion, and the length adjusting portion.

8. A transcatheter valve replacement device according to claim 7, wherein attachment points between said portions exhibit mechanical play in the absence of rigid connections, such that, the devices bends at the attachment points.

9. A transcatheter valve replacement device according to claim 4, wherein the bendable portion of the valve frame is configured as a length adjusting portion comprising an open mesh structure defining stent cells between adjacent stent portions.

10. A transcatheter valve replacement device according to claim 9, wherein the bendable and length adjusting portions can be configured to have a series of vertical links between the adjacent stent portions.

11. A transcatheter valve replacement device according to claim 10, wherein the vertical links extend around the circumference of the stent, between pairs of rows of the stent cells, to accommodate bending of the valve frame.

12. A transcatheter valve replacement device according to claim 9, wherein adjacent stent portions are connected by a pliable sealing component selected from the group consisting of fabric, PET, PTFE, polyester cloth and pericardial tissues to accommodate bending between the stent portions.

13. A transcatheter valve replacement device according to claim 4, wherein the middle portion has a height dimension measured along a major axis of the valve frame and between opposite ends of the valve frame, wherein the height dimension is between about 0.5 to about 1.5 times a radial length of displaced diseased leaflets originally present in the installation site.

14. A transcatheter valve replacement device according to claim 4, comprising only a middle portion of the valve frame and at least one prosthetic leaflet thereon in the absence of an upper flared portion, a lower flared portion, a bendable portion, and a length adjusting portion.

15. A transcatheter valve replacement device according to claim 4, wherein the middle portion defines prong attachment holes to facilitate the attachment of the at least one prong to the valve frame.

16. A transcatheter valve replacement device according to claim 1, wherein the leaflet and the at least one prong are positioned within the valve frame and at the installation site such that during an opening action of a prosthetic valve proximate the installation site, movement of the leaflet clears all portions of the prosthetic valve during the opening.
